(19) United States
(12) Patent Application Publication
(10) Pub. No.: US 2015/0343560 A1
(43) Pub. Date: Dec. 3, 2015

(54) APPARATUS AND METHOD FOR CONTROLLED LASER HEATING

(71) Applicants: Richard G. Pettit, Fruit Heights, UT (US); Ripudaman Singh, Cromwell, CT (US)

(72) Inventors: Richard G. Pettit, Fruit Heights, UT (US); Ripudaman Singh, Cromwell, CT (US)

(73) Assignee: FRACTURELAB, LLC, Fruit Heights, UT (US)

(21) Appl. No.: 14/293,537

(22) Filed: Jun. 2, 2014

(51) Int. Cl. B23K 26/03; B23K 26/12; G01N 21/88; A61B 18/20; C21D 1/34; G01N 21/55; B23K 26/00; B23K 26/06

(52) U.S. Cl. CPC B23K 26/032; B23K 26/0069; B23K 26/128; B23K 26/0626; A61B 18/20; C21D 1/34; G01N 21/55; G01N 21/8806; A61B 2018/00714

(57) ABSTRACT

The invention pertains to an apparatus and method for controlled laser heating of a body. An optical integrating chamber, with an opening adjacent to the surface of the body, has a first and second aperture. A laser source, produces a beam of known power which is directed through the first aperture and the chamber opening onto the surface. A portion of the power of the laser beam is absorbed by the body, thereby heating it locally, and the remaining portion is substantially reflected back into the chamber. A photodetector samples the reflected light accumulated within the chamber through the second aperture, thereby discerning the total power of the reflected light, and enabling the computation of the absorbed power imparted as heat to the body. This computation is performed by a computer or controller, which also serves as a control feedback mechanism, by which the application of the laser is controlled based on the absorbed power imparted to the body during the heating process. Several embodiments are described, useful for a wide range of potential applications in processing and evaluation of organic and inorganic materials and structures.

### CROSS-REFERENCE TO RELATED APPLICATIONS

Not Applicable.

### BACKGROUND OF THE INVENTION

1. Field of the Invention

The invention pertains to an apparatus and method for controlled laser heating of a substantially solid body. Potential uses include any application which would benefit from the ability to quantify or prescribe the amount of laser power or energy actually imparted to and absorbed within the body, thus including but not limited to laser processing of inorganic materials, such as metals and ceramics, laser processing of organic materials or tissues, including laser surgery, and local laser interrogation of materials to evaluate material properties or damage. Many potential applications will be apparent to one skilled in the art in light of the description of exemplary embodiments that will be given hereafter.

2. Description of the Prior Art

The use of a laser as a heat source is broadly used in many industries, and is finding new applications at an accelerating rate. Generally, applications require a degree of control on the amount and distribution of laser energy used in the process to achieve a predetermined outcome. Too much or too little energy imparted to the process can negatively impact process quality. Typical process control methods most commonly involve prescribing external parameters such as the intensity or optical power of the laser, the spot size and shape, the exposure time, which may include continuous or pulsed operation, and a feed rate of the laser spot relative to the work piece where applicable. These settings may be determined based on operator experience or empirical evidence. However, because of material variability, especially with regard to the reflectivity of the material, the actual thermal profile, phase changes, and other parameters resulting from a process so prescribed will in fact vary, with potential for process failure or poor quality. This is particularly true when the amount of heating is sufficient to change the reflectance of the material in the course of the process, so that the ratio of the absorbed power to the applied optical power varies in an unknown manner during the process.

One attempt to address this kind of process variability has been to employ closed-loop control of the beam intensity, exposure time or feed rate to maintain a prescribed local surface temperature, as measured by a pyrometer or other means. U.S. Pat. No. 4,317,981 is an early example of this is approach. However, using this method, the amount of energy actually imparted to the work piece is not explicitly known or controlled. Also, pyrometric temperature measurement requires a known value of emissivity, which is the mathematical complement of the reflectance, and thus potentially varies during the heating process.

A second approach to address process variability is to cut off the exposure time based on optical monitoring of the physical appearance (color or reflectance) of the material being processed. This is possible when the process completion is naturally signaled by a change in reflectivity with a distinct signature. An example of this in a medical application is given in a study by Jerath et al, Preliminary Results on Reflectance Feedback Control of Photocoagulation In Vivo (IEEE Transactions on Biomedical Engineering, vol. 41, No 2, 1994), where laser-induced retinal lesions in rabbit eyes created using fixed external process parameters (laser intensity, exposure time) were compared with lesions created by cutting of the laser power when the local reflectance of the tissue (under ambient light, with laser shuttered off) reached a predetermined gray scale value as monitored by a digital camera. The latter showed less variability than the former. Nevertheless, this approach failed to gain widespread use in the medical field; laser surgery is still largely controlled by fixed external parameters, and problems with variability in laser surgery outcomes persist.

In a comparable approach, illustrated in U.S. Pat. No. 4,865,683 for a materials processing application associated with transforming a thin layer of amorphous silicon to crystalline silicon, the laser intensity is raised until a drop in the reflected laser light measured by a photodetector is observed, signaling onset of the phase transformation. However, not all heating processes happen to so conveniently signal completion with a recognizable change in reflectivity.

In other applications, the purpose of the laser heating process is to interrogate the material to evaluate material properties or damage, such as in U.S. application Ser. No. 14/173,813, where cyclic laser heating is used to run thermo-mechanical fatigue, crack growth, or creep tests. This class of apparatus and method can also benefit from better controlled heating than can be achieved by prior art methods.

### SUMMARY OF THE INVENTION

The invention encompasses an apparatus and method for controlled laser heating of a body in such a way that the user can quantify or prescribe the amount of laser-induced thermal energy imparted to and absorbed within the body according to a predetermined schedule, regardless of the reflectance, or changes in reflectance of the surface of the body during the heating process.

Applications include laser processing of inorganic materials, such as metals and ceramics, laser processing of organic materials or tissues, including laser surgery, and local interrogation of materials to evaluate material properties or damage.

In the ensuing description, the laser heating process will be described in terms of a laser beam directed at the surface of a body or work piece, with a portion of the optical power being absorbed as thermal power, and the remainder being reflected, as if the materials were perfectly opaque. For materials of limited translucence, such as organic tissue or some polymeric materials, “reflected” light so described may be understood to include light that is reflected, diffusely reflected, backscattered or otherwise re-emitted from the surface in the immediate vicinity of the incident laser spot. The principles of operation described in the following remain applicable so long as the proportion of light transmitted through the body and emitted remotely is sufficiently small to be negligible. For such materials, the absorbed power may be considered equal to the difference between the incident power and the reflected power.

P_{absorbed}=P_{incident}−P_{reflected} (1)

An apparatus for laser heating of a body is thus considered, consisting of a body, having a surface, and an optical integrating chamber, with an opening adjacent to the surface of the body, and a first and second aperture elsewhere on the chamber. A laser source emits a beam of known power directed through the first aperture, which continues through the chamber, and is incident upon the surface of the body exposed by opening of the chamber. As described above, a portion of the optical power of the beam is absorbed by the body, thereby heating it locally, and the remaining portion is substantially reflected back into the chamber.

A photodetector samples the reflected light through the second aperture, by which the total power of the reflected light can be determined based on a calibration of the integrating chamber. The absorbed power can then be calculated by the formula given above.

Using a control feedback mechanism, such as a closed-loop computer system or controller, the application of the laser is controlled based on the absorbed power imparted to the body.

The application of the laser may be controlled by varying the incident laser beam power, the laser pulse duration, and the feed rate of the beam relative to the surface. The first two control modes could be achieved by feedback control to the laser source; the third mode would require feedback to automated equipment that controls the movement of the laser relative to the workpiece, where applicable. A given embodiment may potentially include any combination of these control modes.

In general, by feedback control of the incident laser power, the absorbed thermal power can be imparted to the body according to a predetermined schedule. For many applications, it is useful to configure the controller to keep the absorbed power substantially constant during the heating process, such as for laser heat treating or hardening application with a constant feed rate. For cycled or pulsed operation, such as in laser fatigue testing applications, it is useful to configure the controller to create a sequence of pulses of absorbed power of substantially constant amplitude during said heating process.

Note, however that an energetically similar process could be obtained by holding the incident optical power constant, but varying the feed rate or pulse width to impart the same amount of absorbed energy per unit length of feed, or per pulse.

More generally, the controller may be configured to integrate the absorbed power over time during the heating process; thereby controlling application of the laser based on total absorbed energy, or absorbed energy per pulse. For example, a stationary process could be specified to a constant incident power, with the power shut off when after a specified total energy has been applied to the work piece.

For many applications, it is useful to configure the chamber to be segmented into at least two parts, with a detachable tip that includes the chamber opening, which can be replaced with other tip configurations of predetermined purpose.

For example, integrating chambers are often configured to be spherical, because that shape efficiently amplifies and integrates the light being measured. Indeed a spherical chamber may be a good choice for many applications. But an integrating sphere of useful size may not be suitable for access into fillets or other surface irregularities found in some applications. Some special purpose chamber configurations will now be discussed, which could optionally be configured as interchangeable tips on a common chamber base.

In one such configuration, the integrating chamber is of a substantially teardrop-like shape, with the geometry of the cusp of the teardrop truncated or similarly modified to accommodate the chamber opening, thereby permitting improved access into fillets or depressions in the surface of the body.

In a further variant, the interior surface of a teardrop-shaped integrating chamber is of a terraced configuration in the vicinity of the (albeit truncated) cusp of the teardrop, thereby more efficiently reflecting light back into the more spherical portion of the chamber, and increasing the efficiency or optical gain of the chamber. The terraced configuration is somewhat more difficult to clean, however, so its use may depend on the cleanliness of the anticipated operating environment.

In another configuration, the chamber is configured to extend into a hole or slot, and said opening is to the side, thereby enabling controlled laser heating of the side of said hole or slot. For shallow holes, it is possible to apply the laser to the surface through the opening directly from a first aperture on the opposite side.

For deeper holes, this is not practical. In this case the beam can be applied indirectly by way of a mirror. That is, a portion of the chamber surface in the vicinity of the opening is of high specular reflectance, thereby assisting transmission of incident or reflected radiation around the corner between the opening and the remainder of the chamber. Such a configuration also lends itself readily to be further configured with a detachable interchangeable tip for either shallow or deep holes.

It is also useful to include additional apertures in the integrating chamber, with corresponding devices including one or more of the following: a pyrometer, an infrared camera, a visible light source of mixed spectrum, and a visible light camera. Each device has optical access to the heated portion of the surface through the aperture provided to further interrogate the surface during operation.

By adding pyrometric capability, the temperature evolution at a point on the surface is known simultaneously with the history of the absorbed thermal power, permitting the estimation of the thermal physical properties of the body. For a laser spot of known characteristic diameter r and flux profile, irradiating a semi-infinite body of material at initial temperature T₀ with constant absorbed power Q until a maximum steady-state value Tₘₐₓ is approached (typically at the center of the spot), the thermal conductivity is given by

$$k = \frac{CQ}{r ( {T}_{ma x} - {T}_{0} )} \qquad ( 2 )$$

The parameter C is constant for a given flux profile, is known for common profiles (C=1/π for a uniform flux profile of radius r), and can be determined numerically for others by means common to the art. In many practical situations, the body is many times the size of the laser spot, and thus effectively semi-infinite for the purpose of this calculation. The calculation also assumes that k is constant at temperatures between T and Tₘₐₓ, which is a good approximation for modest thermal excursions. By similar means, other thermal properties can likewise be deduced from the same information.

With use of an infrared camera with pyrometric capability, the evolution of the local temperature distribution can be obtained, rather than just at a point, enabling a more thorough interrogation of the thermal response of the body. This also guarantees that the point of maximum temperature will not be missed.

By adding a light source of mixed spectrum, and two visible wavelength cameras operable within the spectral range of the light source, it is possible to interrogate the surface deflections using well-known digital image correlation (DIC) technology. This also typically requires application of a fine high-contrast speckle pattern to the surface over the domain being interrogated. By this means, crack growth arising from cyclic thermal stresses, or deflections due to creep can also be observed and evaluated.

Here we note that physical properties like thermal conductivity have been observed to change with advancing exposure to mechanical or thermo-mechanical fatigue, even before the appearance of observable cracks. Typically, these changes would be expected to appear most critically at stress concentrations due to irregular surface features. Inspection of structural members to assess otherwise invisible fatigue damage could thus be performed by interrogating physical properties using low-level (non-damaging) controlled laser heating.

Many potential uses for the controlled laser heating apparatus and method are thus encompassed in the present invention which include, but are not limited to those mentioned above.

In addition to the apparatus described above and hereafter, the invention encompasses the laser heating method, or process described herein. In summary, the process includes directing a laser beam of known incident optical power onto the surface of a body, measuring the power of the laser light reflected from the surface using an integrating sphere and a photodetector, calculating the absorbed power (as the difference between the incident and reflected power), and varying the application of the laser using a control feedback mechanism based on the total absorbed power imparted to the body.

Further, the process includes use of all embodiments as described.

As can be seen, many other useful embodiments and applications of the controlled laser heating technology described could be devised by one skilled in the art.

### BRIEF DESCRIPTION OF THE DRAWINGS

The invention is described by way of example with reference to embodiments that are illustrated in the figures, but without thereby restricting the general object of the invention. Closely related figures have the same number, but different alphabetic suffixes.

FIG. 1A shows a schematic representation of an apparatus for controlled laser heating of a body in cross section. FIG. 1B shows an embodiment with a tear-drop-shaped optical integrating chamber for reaching into fillets and depressions in the workpiece. FIG. 1C illustrates an embodiment with a tear-drop-shaped chamber, further configured with a terraced configuration for improved optical gain. FIG. 1D shows an embodiment configured to provide access into a hole or slot.

FIG. 2 shows a schematic representation of an apparatus for controlled laser heating of a body, with additional apertures and mounted instrumentation to enable further interrogation of the surface of the workpiece during operation.

FIG. 3 shows a schematic illustration of a process control loop for controlled laser heating of a body.

### DESCRIPTION OF EXEMPLARY EMBODIMENTS

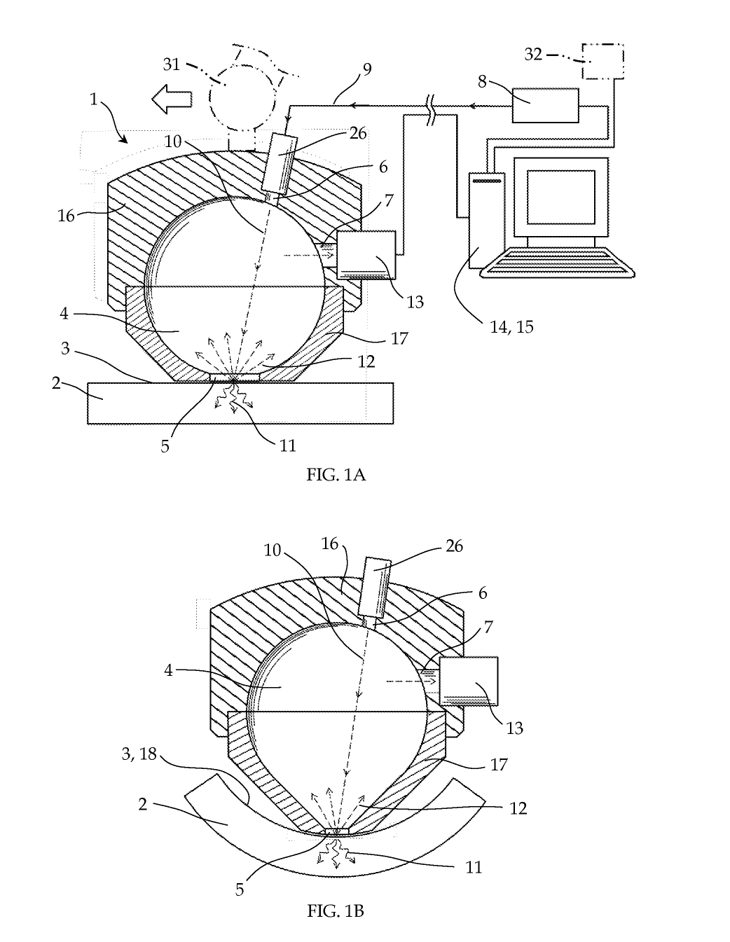

FIG. 1A, shows a schematic representation of an apparatus 1 for controlled laser heating of a body 2, including a body 2 having a surface 3, an optical integrating chamber 4, with an opening 5 adjacent to the surface 3 of the body 2, and a first aperture 6 and second aperture 7. A laser source 8, produces a beam 10 of known power which is directed through the first aperture 6 and the chamber opening 5 onto the surface 3. For illustration, the beam is carried to the chamber by a fiber optic cable 9, which is mounted to the chamber wall 16 by attachment 26. A portion 11 of the power of the laser beam 10 is absorbed by the body, thereby heating it locally, and the remaining portion 12 is substantially reflected back into the chamber 4.

A photodetector 13 samples the reflected light accumulated within the chamber 4 through the second aperture 7, thereby discerning the total power of the reflected light 12, and enabling the computation of the absorbed power 11 imparted as heat to the body 2. This computation is performed by a computer or controller 14, which also serves as a control feedback mechanism 15, by which the application of the laser 10 is controlled based on the absorbed power 11 imparted to the body during the heating process.

For some uses, such as laser heat treatment or hardening, or other surface processing, the laser beam 10, along with the chamber 4 may optionally be configured to move relative to the surface 3 of the body 2 being processed. Such movement, or feed, is typically automated using means common to the art, such as robotic arm 31 and its controller 32 are portrayed schematically. For other applications such as thermal fatigue testing, the laser 10 may be pulsed or cycled. For applications like laser shock peening, both feed and cycling may occur. For control purposes, the application of the laser 10 may be adjusted by varying heating parameters such as the power of the incident laser beam 10, the laser pulse duration, and the feed rate of the beam 10 relative to the surface 3, based on said absorbed power 11.

In general, by feedback control of the laser source 8, and thus the power of the incident laser beam 10, absorbed thermal power 11 can be imparted to the body according to a predetermined schedule. For many applications, it is useful to configure the controller 15 to keep the absorbed power 11 substantially constant during said heating process, such as a for a laser heat treating or hardening application with a constant feed rate. For cycled or pulsed operation, such as in laser fatigue testing applications, it is useful to configure the controller to create a sequence of pulses of absorbed power 11 of substantially constant amplitude during said heating process.

An energetically equivalent process can be obtained by holding the incident optical power constant, but varying the feed or pulse width to impart the same amount of absorbed energy per unit length of feed, or per pulse. For the robot arm 31 illustrated, control of feed rate can be implemented by connecting the laser process controller 15 to the robot arm controller 32 to command the feed rate.

More generally, the control feedback mechanism 14 based on the absorbed power 11 may be configured to integrate said absorbed power over time during the heating process; thereby controlling application of the laser 10 based on total absorbed energy, or absorbed energy per pulse.

In FIG. 1A the chamber is shown segmented into at least two parts, with a detachable tip 17 that includes the chamber opening 5, which can be replaced with other tip configurations of predetermined purpose.

Some examples of other chamber configurations with a similar arrangement having different interchangeable tips 17 are shown in FIGS. 1B, 1C, and 1D. The laser source and controller are omitted in these figures. In FIG. 1B, the integrating chamber is of a substantially teardrop-like shape, with the geometry of the cusp of the teardrop substantially truncated to accommodate the opening 5, thereby permitting improved access into a fillet 18 or other depression in the surface 3.

While a chamber 4 of tear-drop shape is useful for reaching into a surface irregularity or fillet 18, the optical gain of such a chamber 4 may be somewhat reduced compared to the spherical configuration shown above. As shown in FIG. 1C, it is useful in some applications to configure the interior surface of the chamber 4 to a terraced geometry 20 in the vicinity of the cusp or otherwise extended portion of the chamber 4 as shown in FIG. 1C, thereby more efficiently re-reflecting accumulated light 33 back into the more spherical portion of the chamber 4, and increasing the gain of the chamber 4.

In FIG. 1D, the chamber 4 is configured to extend into a hole or slot 19, and the opening 5 is to the side, thereby enabling controlled laser heating of the side of the hole or slot 19. The embodiment illustrated is further configured with a portion of the chamber surface in the vicinity of the opening 5 being a mirror 21 of high specular reflectance, for the wavelength of the laser radiation, thereby assisting transmission of incident light 10 or reflected light 12 around the corner between the opening 5 and the remainder of the chamber 4.

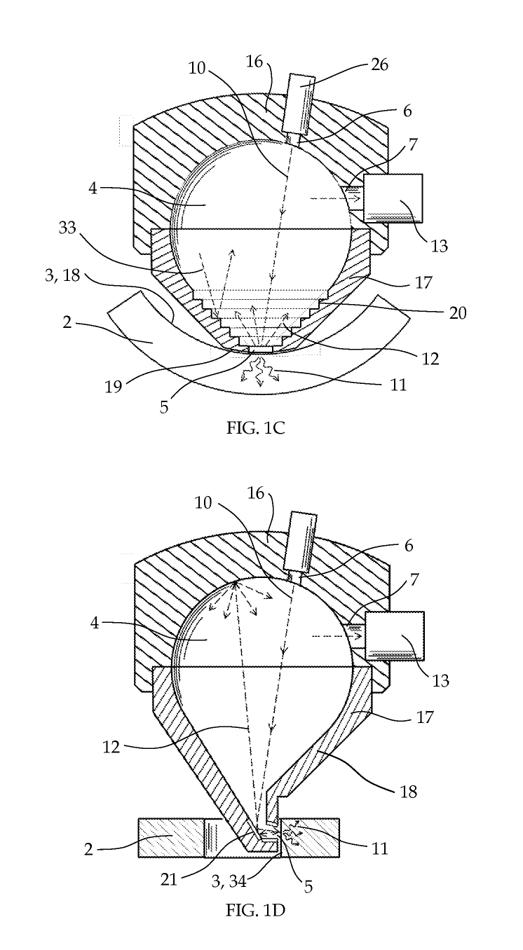

FIG. 2 shows a schematic representation of an exemplary apparatus 1 for controlled laser heating, further configured with at least one additional aperture 34 in the integrating chamber 4, and instrumented with one or more of the following devices: a pyrometer 22, an infrared camera 23, a visible light source of mixed spectrum 24, and a visible light camera 25; wherein each of the devices has optical access through the at least one additional aperture to the heated portion of the surface 3 of the body 2, thereby enabling further interrogation of said surface 3 during operation.

In particular, the apparatus shown includes an infrared camera 23 with pyrometric capability (thus also serving as a pyrometer 22), permitting monitoring of the evolution of the thermal distribution resulting from the imparted thermal input 11 by way of the associated infrared radiation 35. This additional information enables estimation of local physical properties such as the thermal conductivity as described earlier. Also shown is a visible light source of mixed spectrum 24, and two cameras 25 operable within the spectral range of the light source 24, thereby enabling interrogation of the surface deflections using DIC. This enables monitoring of the thermo-mechanical strains, crack growth, and deflections due to creep.

It should be mentioned that for the laser-heating and DIC components to function independently in the same chamber 4, the visible mixed-spectrum light source 24 should not include the wavelength of the laser 10, and should be excluded from the photodetector 13. Likewise, the wavelength of the laser 10 should be out of the range of, or filtered out of the visible light cameras 25 used in the digital image correlation. This can be accomplished by judicious selection of the wavelength range of operation of these components, or by using various filter arrangements (not shown) common to the art. Interference of these devices with the pyrometric sensors, if any, may be averted in the same manner.

Fatigue crack growth and creep measurements associated with controlled cyclic heating would be locally damaging to the body, which in this case may be a test specimen. However, changes in the local physical properties, observed in a less severe manner using non-damaging thermal excursions of controlled laser heating can be useful in assessing the remaining life of structural members non-destructively.

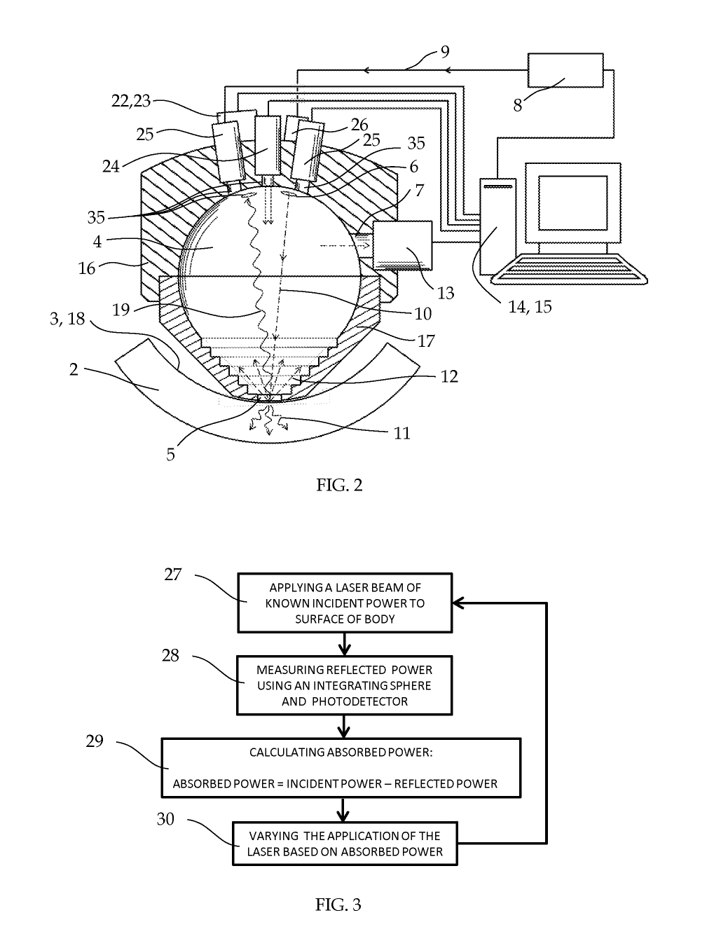

In addition to the apparatus described above, the invention encompasses the laser heating process described herein, and illustrated in FIG. 3. In summary, the process includes a control loop including the steps of applying 27 a laser beam of known incident optical power to the surface of a body, measuring 28 the power of the laser light reflected from the surface using an integrating sphere and a photodetector, calculating 29 the absorbed power (as the difference between the incident and reflected power), and varying 30 the application of the laser using a control feedback mechanism based on the total absorbed power imparted to the body.

Further, variants of the process include use of all embodiments as described.

Many potential uses for the controlled laser heating apparatus and process are thus encompassed in the present invention which include, but are not limited to the following: to control a laser heat-treating process, to control a laser hardening process, to control a laser shock-peening process, to control heating of biological tissue during a laser surgery, to evaluate the thermal conductivity of a body, to evaluate the creep properties of a body, to evaluate the thermo-mechanical fatigue properties of a body, to evaluate the thermo-mechanical fatigue crack growth properties of a body, and to interrogate changes in the physical properties of a body associated with fatigue damage accumulating within the body, thereby assessing the remaining life of the body.

Although the present invention has been described in considerable detail with reference to certain preferred versions thereof, alternate configurations and arrangements can be easily devised by one skilled in the art. Therefore, the spirit and scope of the appended claims should not be limited to the description of the preferred versions contained herein. The reader's attention is directed to all papers and documents which are filed concurrently with this specification and which are open to public inspection with this specification, and the contents of all such papers and documents are incorporated herein by reference. All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

### LIST OF REFERENCE SYMBOLS

1 Apparatus for controlled heating of a body
2 Body or workpiece
3 Surface of body
4 Optical integrating chamber
5 Chamber opening
6 First aperture
7 Second aperture
8 Laser source
9 Fiber-optic cable
10 Laser beam
11 Thermal power absorbed into body from laser beam
12 Reflected light
13 Photodetector
14 Control feedback mechanism
15 Computer or controller
16 Chamber wall
17 Removable tip
18 Fillet
19 Hole or slot
20 Terraced surface within chamber
21 Mirror of high specular reflectance
22 Pyrometer
23 Infrared camera
24 Visible light source of mixed spectra
25 Visible light camera
26 Optical fiber attachment
27 Step of applying laser beam of known incident power to surface of body
28 Step of measuring reflected power using an integrating sphere and photodetector.
29 Step of calculating absorbed power
30 Step of varying the application of the laser based on absorbed power
31 Robotic arm
32 Feed rate controller
33 Accumulated light re-reflected back into chamber
34 Additional aperture
35 Infrared signal radiating from heated surface

The invention claimed is:

1. An apparatus for controlled laser heating of a body, comprising:
(a) a body having a surface; and
(b) an optical integrating chamber, with an opening adjacent to the surface of the body, and a first and second aperture; and
(c) a laser source, with a beam of known power directed through said first aperture and said opening onto said surface, a portion of the power of said beam being absorbed by the body, thereby heating it locally, and the remaining portion being substantially reflected back into the chamber; and
(d) a photodetector that samples said reflected light through said second aperture, thereby discerning the total power of the reflected light, and enabling the computation of the absorbed power; and
(e) a control feedback mechanism by which the application of the laser is controlled based on the absorbed power imparted to the body during the heating process.

2. The apparatus according to claim 1 wherein said application of the laser controlled by said feedback mechanism is configured to be controlled by varying one or more parameters selected from the list consisting of:
(a) the incident laser beam power,
(b) the laser pulse duration, and
(c) the feed rate of the beam relative to the surface.

3. The apparatus according to claim 2 wherein said control feedback mechanism is configured to control said application of said laser by controlling said incident laser power to keep said absorbed power substantially constant during said heating process.

4. The apparatus according to claim 2 wherein said control feedback mechanism is configured to control said application of said laser by controlling said incident laser power to create a sequence of pulses of absorbed power of substantially constant amplitude during said heating process.

5. The apparatus according to claim 1 wherein said control feedback mechanism based on said absorbed power is configured to integrate said absorbed power over time during the heating process; thereby controlling application of the laser based on total absorbed energy, or absorbed energy per pulse.

6. The apparatus of claim 1 wherein the chamber is segmented into at least two parts, with a detachable tip that includes the chamber opening, which can be replaced with other tip configurations of predetermined purpose.

7. The apparatus according to claim 1 wherein said integrating chamber is of a substantially teardrop-like shape, with the geometry of the cusp of the teardrop substantially truncated to accommodate said opening, thereby permitting improved access into fillets or depressions in said surface.

8. The apparatus according to claim 7 wherein the interior surface of said integrating chamber is of a terraced configuration in the vicinity of the cusp, thereby more efficiently reflecting light back into the more spherical portion of the chamber, and increasing the efficiency of the chamber.

9. The apparatus according to claim 1 wherein said chamber is configured to extend into a hole or slot, and said opening is to the side, thereby enabling controlled laser heating of the side of said hole or slot.

10. The apparatus of claim 9 wherein a portion of the chamber surface in the vicinity of said opening is configured to be of high specular reflectance, thereby assisting transmission of incident or reflected radiation around the corner between the opening and the remainder of the chamber.

11. The apparatus according to claim 1 further comprising:
(a) at least one additional aperture in said chamber, and;
(b) one or more devices selected from the following list:
(1) a pyrometer,
(2) an infrared camera,
(3) a light source of mixed spectrum, and
(4) a visible light camera;
wherein each of said devices has optical access through said at least one additional aperture to the heated portion of said body, thereby enabling further interrogation of said surface during operation.

12. The apparatus of claim 11, wherein said at least one device includes an infrared camera with pyrometric capability.

13. The apparatus of claim 11, wherein said at least one device includes a light source of mixed spectrum, and two cameras operable within the spectral range of the light source, thereby enabling interrogation of the surface deflections using digital image correlation.

14. The apparatus of claim 1, applied to one or more purposes selected from a list including the following:
(a) to control a laser heat-treating process,
(b) to control a laser hardening process,
(c) to control a laser shock-peening process,
(d) to control heating of biological tissue during a laser surgery,
(e) to evaluate the thermal conductivity of said body,
(f) to evaluate the creep properties of said body,
(g) to evaluate the thermo-mechanical fatigue properties of said body,
(h) to evaluate the thermo-mechanical fatigue crack growth properties of said body, and
(i) to interrogate changes in the physical properties of said body associated with fatigue damage accumulating within said body, thereby assessing the remaining life of the body.

15. A laser heating process comprising:
(a) directing a laser beam of known incident optical power onto the surface of a body; and
(b) measuring the power of the laser light reflected from the surface using an integrating sphere and a photodetector; and
(c) calculating the absorbed power, as the difference between the incident and reflected power; and
(d) varying the application of the laser using a control feedback mechanism based on the total absorbed power imparted to the body.

16. The process of claim 15, wherein said varying of the application of said laser includes varying one or more heating parameters selected from the list consisting of:
(a) the incident laser beam power, and
(b) the feed rate of the beam relative to the surface, and
(c) the laser pulse duration.

17. The process of claim 15, wherein said varying of the application of said laser comprises varying said incident laser beam power, and is controlled to keep said absorbed power substantially constant during said process.

18. The process of claim 15, wherein said varying of the application of said laser comprises varying said incident laser beam power, and is controlled to apply pulses of said absorbed power of substantially constant amplitude during said process.

19. The process of claim 15 wherein said control feedback mechanism based on said absorbed power is configured to integrate said absorbed power over time during the heating process, thereby controlling application of the laser based on total absorbed energy, or absorbed energy per pulse.

20. The process of claim 15 wherein said process is applied to one or more purposes selected from a list including the following:
(a) to control a laser heat-treating process,
(b) to control a laser hardening process,
(c) to control a laser shock-peening process,
(d) to control heating of biological tissue during a laser surgery,
(e) to evaluate the thermal conductivity of said body,
(f) to evaluate the creep properties of said body,
(g) to evaluate the thermo-mechanical fatigue properties of said body,
(h) to evaluate the thermo-mechanical fatigue crack growth properties of said body, and
(i) to interrogate changes in the physical properties of said body associated with fatigue damage accumulating within said body, thereby assessing the remaining life of said body.
